(19) United States
(12) Patent Application Publication
(10) Pub. No.: US 2014/0187965 A1
(43) Pub. Date: Jul. 3, 2014

(54) Stepped Banded Connector for Intravascular Ultrasound Devices

(71) Applicant: Volcano Corporation, San Diego, CA (US)

(72) Inventor: Michael Reiter, San Diego, CA (US)

(21) Appl. No.: 14/137,649

(22) Filed: Dec. 20, 2013

Related U.S. Application Data
(60) Provisional application No. 61/747,468, filed on Dec. 31, 2012.

(51) Int. Cl. A61B 8/00

(52) U.S. Cl. CPC A61B 8/445
USPC 600/467; 439/660

(57) ABSTRACT

A male and female connectors and a method for providing electrical connection between a patient interface module (PIM) and an intravascular sensing device carrying more than two signal leads. A four-electrical-contacts male connector includes three elongated hollow tubular male bodies concentrically disposed around one another in an expanded telescopic fashion to define three stepped banded outer surfaces, where one electrical contact is formed on an inner surface of the innermost male body, and the rest on the stepped bands. The female connector includes three spaced-apart hollow tubular female bodies concentrically disposed around one another and a shaft disposed within the innermost female body, being sized and configured, respectively, to mate with the three stepped bands and a central cavity defined by the innermost male body of the male connector.

### CROSS-REFERENCE TO RELATED APPLICATION

The present application claims the benefit of the filing date of provisional U.S. Patent Application No. 61/747,468 filed Dec. 31, 2012. The entire disclosure of this provisional application is incorporated herein by this reference.

### TECHNICAL FIELD

The present disclosure relates generally to an intravascular ultrasound (IVUS) imaging catheter, more particularly a connector providing a mechanical and electrical connection between an IVUS device and a patient interface module (PIM).

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. IVUS imaging uses ultrasound echoes to form a cross-sectional image of the vessel of interest. Typically, the ultrasound transducer on an IVUS catheter both emits ultrasound pulses and receives the reflected ultrasound echoes. The ultrasound waves pass easily through most tissues and blood, but they are partially reflected by discontinuities arising from tissue structures (such as the various layers of the vessel wall), red blood cells, and other features of interest. The IVUS imaging system, which is connected to the IVUS catheter by way of a patient interface module (PIM), processes the received ultrasound echoes to produce a cross-sectional image of the vessel where the catheter is located.

There are two types of IVUS catheters in common use today: solid-state and rotational, with each having advantages and disadvantages. Solid-state, or phased array IVUS catheters use an array of ultrasound transducers (typically 64) distributed around the circumference of the catheter and connected to an electronic multiplexer circuit. The multiplexer circuit selects array elements for transmitting an ultrasound pulse and receiving the echo signal. By stepping through a sequence of transmit-receive pairs, the solid-state IVUS system can synthesize the effect of a mechanically scanned transducer element, but without moving parts. Since there is no rotating mechanical element, the transducer array can be placed in direct contact with the blood and vessel tissue with minimal risk of vessel trauma and the solid-state scanner can be wired directly to the imaging system with a simple electrical cable and a standard detachable electrical connector.

In the typical rotational IVUS catheter, a single ultrasound transducer element fabricated from a piezoelectric ceramic material is located at the tip of a flexible driveshaft that spins inside a plastic sheath inserted into the vessel of interest. The transducer element is oriented such that the ultrasound beam propagates generally perpendicular to the axis of the catheter. The fluid-filled sheath protects the vessel tissue from the spinning transducer and driveshaft while permitting ultrasound signals to freely propagate from the transducer into the tissue and back. As the drive shaft rotates (typically at 30 revolutions per second), the transducer is periodically excited with a high voltage pulse to emit a short burst of ultrasound. The same transducer then listens for the returning echoes reflected from various tissue structures, and the IVUS imaging system assembles a two dimensional display of the vessel cross-section from a sequence of several hundred of these ultrasound pulse/echo acquisition sequences occurring during a single revolution of the transducer.

While existing rotational IVUS catheters deliver useful diagnostic information, there is a constant need for enhanced image quality to provide more valuable insight into the vessel condition. One problem noted for deterring further improvement in image quality in rotational IVUS imaging is that the electrical impedance of the transducer is too high to efficiently drive the electrical cable connecting the transducer to the IVUS imaging system by way of the PIM. To solve the problem, attempts have been made for improving transmit electronics or other signal processing through devising more compact and efficient circuit architecture and electrical interface for a polymer piezoelectric micro-machined ultrasonic transducer used in an intravascular ultrasound system. One of the recent developments from such attempts is the advance of a rotational IVUS catheter that has a circuit architecture using four, instead of the conventional two, wire interface to the PIM. Such new IVUS catheters using two pairs of signal leads may require use of an electrical cable carrying four signal lines, and by driving the electrical cable connecting the transducer to the IVUS imaging system more efficiently, can offers a greater resolution than the conventional two signal leads catheter.

As such, such four signal leads IVUS catheters require a special PIM connector assembly, comprising male and female connectors, which can establish electromechanical connection between the IVUS catheters and a PIM through the coupling of four electrical channels or contacts.

Thus, there remains a need for improved connection assemblies between sensing catheters and external components receiving data from the sensors.

### SUMMARY

Embodiments of the present disclosure provide a compact and efficient circuit architecture and electrical interface to a polymer piezoelectric micro-machined ultrasonic transducer used in an intravascular ultrasound system. In particular, the present disclosure provides a catheter with a stepped band connection assembly that can accommodate three or more conductive elements.

In one aspect of the present disclosure, a sensing catheter having a male electrical connector is provided. The male electrical connector comprises at least two elongated hollow tubular male bodies having respective proximal and distal ends. In some embodiment, the two hollow tubular male bodies may have a general configuration of a hollow cylindrical shell. The two tubular male bodies are concentrically and sequentially disposed around one another in an expanded telescopic fashion to define stepped banded respective outer surfaces, respectively extending from respective proximal ends toward respective distal ends. The innermost male body defines an elongated cavity enclosed by its inner surface and extending lengthwise between its proximal and distal ends. Some portions on the respective stepped banded outer surfaces of the three tubular male bodies adjacent respective proximal ends, and a portion of the inner surface of the innermost tubular male body adjacent its proximal end are conductive to form three electrical contacts. The electrical contacts are electrically insulated from one another.

In an embodiment, the electrical insulation among the electrical contacts may be achieved by dielectric layers concentrically disposed among the tubular male bodies. Also, in an embodiment, the tubular male bodies may define similar stepped banded respective outer surfaces at the respective distal ends as well, respectively extending toward the respective proximal ends.

In another aspect of the present disclosure, a female connector for a patient interface module is provided. The female connector comprises: a proximal portion having mutually insulated electrical contacts and a distal portion configured to be connected and electrically coupled to a patient interface module (PIM). The proximal portion is sized and configured to mate, and be electrically coupled, with a two-electrical-contacts male connector via two of four electrical contacts, or with a four-electrical-contacts male connector via four electrical contacts.

In one embodiment, the proximal portion of the female connector may comprise: three spaced-apart hollow tubular female bodies concentrically and sequentially disposed around one another, and a shaft concentrically disposed within the innermost tubular female body. In one embodiment, each of the three tubular female bodies has a general configuration of a hollow cylindrical shell. The three tubular female bodies are sized and configured such that their three respective inner surfaces can mate with three respective stepped banded outer surfaces of a four-electrical-contacts male connector where the outer surfaces are defined by disposing three elongated tubular male bodies concentrically and sequentially around one another in an expanded telescopic fashion. The shaft is sized and configured to mate with an elongated cavity defined by an inner surface of the innermost tubular male body of the four-electrical-contacts male connector. The shaft and the innermost tubular female body are further sized and configured such that the inner surface of the innermost tubular female body and the outer surface of the shaft can mate, respectively, with outer and inner surfaces of an elongated hollow tubular male body of a two-electrical-contacts male connector.

In an embodiment, one of the four electrical contacts is formed on an outer surface of the shaft, and the rest are formed on the respective inner surfaces of the three tubular female bodies. In an embodiment, one or more of the three tubular female bodies may be slit at a portion for providing intimate engagement with the male connector.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1A is a schematic illustration of an intravascular ultrasound (IVUS) imaging system, according to some embodiments.

FIG. 1B is a cross-sectional side view of a distal portion of a catheter used in an IVUS imaging system, according to some embodiments.

FIG. 2 is a block diagram of a Patient Interface Module (PIM) for use in an IVUS imaging system, according to some embodiments.

FIG. 3 is an exploded view of a proximal connection assembly of a catheter and a connection assembly of a PIM according to one embodiment

FIG. 4 is a perspective view of a proximal connection assembly of a catheter according to one embodiment.

FIG. 5 is a partial perspective view of the connection assembly of FIG. 4.

FIG. 6 is a partial cross-sectional side view of the connection assembly of FIG. 5.

FIG. 7 is a partial perspective view of a PIM connection assembly.

FIG. 8 is a partial cross-sectional perspective view of the PIM connection assembly of FIG. 7.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

In embodiments of an IVUS catheter disclosed herein, an ultrasound transducer assembly is located at the tip of a flexible driveshaft that spins inside a plastic sheath inserted into the vessel of interest. The transducer assembly includes components oriented such that an ultrasound beam produced by the component propagates generally perpendicular to the axis of the catheter. A fluid-filled sheath protects the vessel tissue from the spinning transducer and driveshaft while permitting ultrasound signals to freely propagate from the transducer into the tissue and back. As the driveshaft rotates (typically at 30 revolutions per second), the transducer is periodically excited with a high voltage pulse to emit a short burst of ultrasound. The same transducer then listens for the returning echoes reflected from various tissue structures, and the IVUS imaging system assembles a two dimensional display of the vessel cross-section from a sequence of several hundred of these pulse/acquisition cycles occurring during a single revolution of the transducer.

In a rotational IVUS catheter, the ultrasound transducer may be a piezoelectric ceramic element with low electrical impedance capable of directly driving an electrical cable connecting the transducer to the imaging system hardware. In this case, a single pair of electrical leads (or coaxial cable) can be used to carry the transmit pulse from the system to the transducer and to carry the received echo signals from the transducer back to the imaging system by way of a patient interface module (“PIM”) where echo signals can be assembled into an image. In embodiments where the catheter driveshaft and transducer are spinning (in order to scan a cross-section of the artery) and the imaging system hardware is stationary, an electromechanical interface couples the electrical signal to a rotating junction. In rotational IVUS imaging systems, this may be achieved by using a rotary transformer, slip rings, rotary capacitors, etc.

In some embodiments, an IVUS catheter may include a plurality of transducer components in a static configuration, forming a phased-array transducer assembly.

Reference will now be made to a particular embodiments of the concepts incorporated into an intravascular ultrasound system. However, the illustrated embodiments and uses thereof are provided as examples only. Without limitation on other systems and uses, such as but without limitation, imaging within any vessel, artery, vein, lumen, passage, tissue or organ within the body. While the following embodiments may refer to a blood vessel and a blood vessel wall for illustrative purposes, any other tissue structure may be envisioned to be imaged according to methods disclosed herein. More generally, any volume within a patient's body may be imaged according to embodiments disclosed herein, the volume including vessels, cavities, lumens, and any other tissue structures, as one of ordinary skill may recognize. Still further, while the distal sensor assembly is illustrated as an ultrasound transducer assembly, it will be appreciated that the assembly can include additional or alternative sensing elements such as for example, but without limitation, pressure sensors, flow sensors, infrared sensors, photo sensors, acoustic sensors, etc.

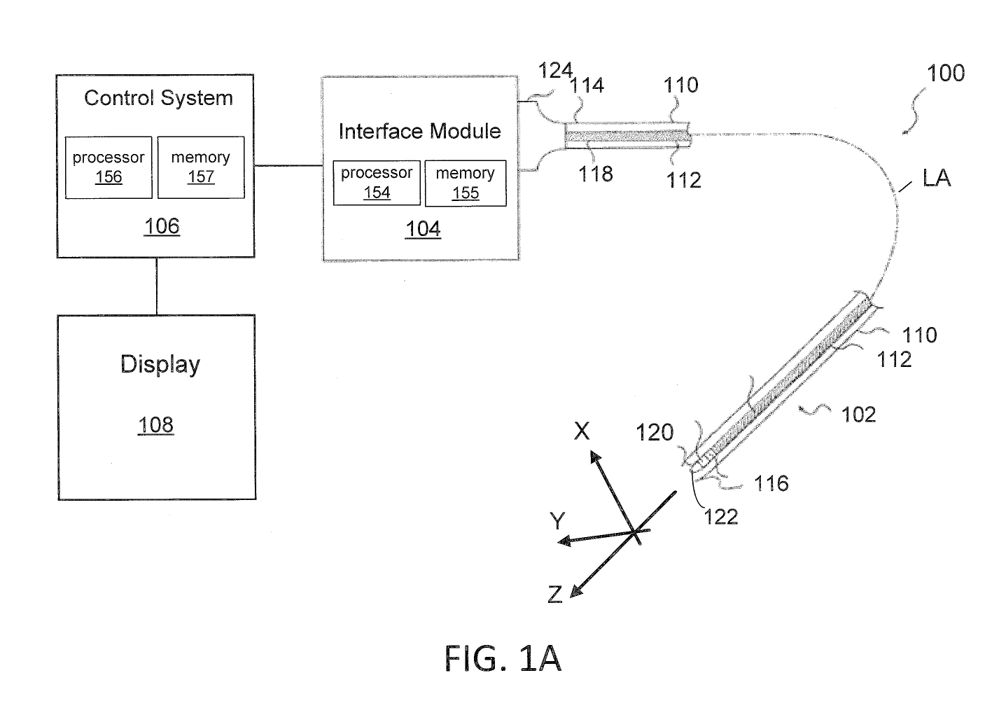

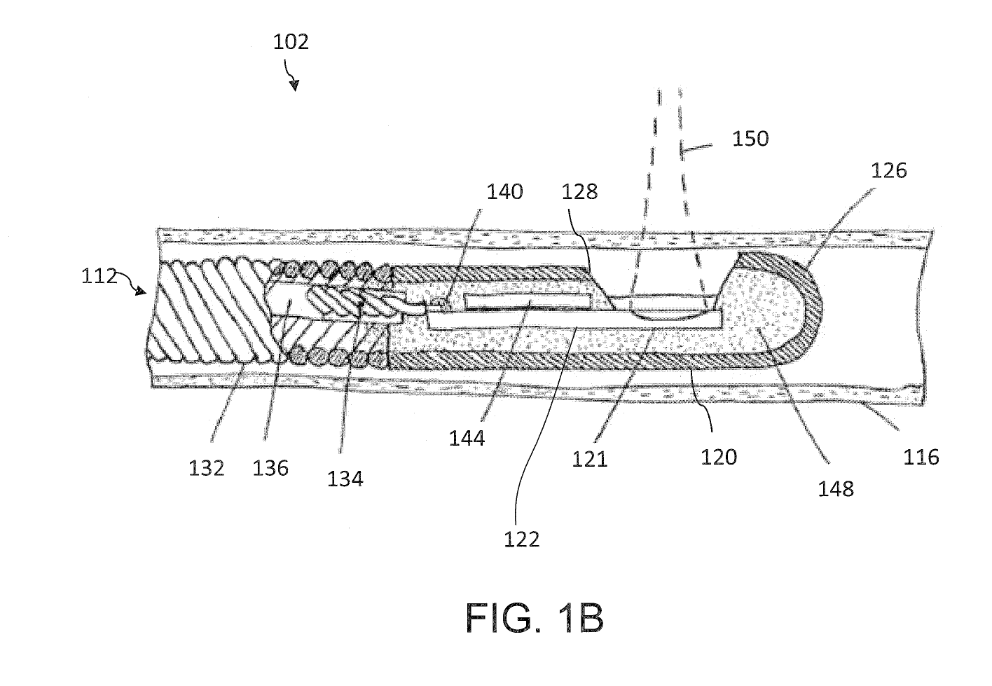

FIG. 1A is a schematic illustration of an intravascular ultrasound (IVUS) imaging system 100, according to some embodiments. IVUS imaging system 100 includes an IVUS catheter 102 coupled by a patient interface module (PIM) 104 to an IVUS control system 106. Control system 106 is coupled to a monitor 108 that displays an IVUS image (such as an image generated by IVUS system 100).

In some embodiments, catheter 102 is a rotational IVUS catheter, which may be similar to a Revolution® Rotational IVUS Imaging Catheter available from Volcano Corporation and/or rotational IVUS catheters disclosed in U.S. Pat. No. 5,243,988 and U.S. Pat. No. 5,546,948, both of which are incorporated herein by reference in their entirety, for all purposes. In some embodiments, catheter 102 may be a stationary component.

Catheter 102 includes an elongated, flexible catheter sheath 110 (having a proximal end portion 114 and a distal end portion 116) shaped and configured for insertion into a lumen of a blood vessel (not shown). In some embodiments, IVUS system 100 may be used for neurological evaluations in blood vessels in the brain, and for renal denervation in blood vessels in the kidney. A longitudinal axis LA of catheter 102 extends between the proximal end portion 114 and the distal end portion 116. Catheter 102 is flexible such that it can adapt to the curvature of the blood vessel during use. In that regard, the curved configuration illustrated in FIG. 1A is for exemplary purposes and in no way limits the manner in which catheter 102 may curve in other embodiments. Generally, catheter 102 may be configured to take on any desired straight or arcuate profile when in use.

In some embodiments an imaging core 112 extends within sheath 110. Accordingly, in some embodiments imaging core 112 may be rotated while sheath 110 remains stationary. Imaging core 112 has a proximal end portion 118 disposed within the proximal end portion 114 of sheath 110 and a distal end portion 120 disposed within the distal end portion 116 of sheath 110. The distal end portion 116 of sheath 110 and the distal end portion 120 of imaging core 112 are inserted into the vessel of interest during operation of the IVUS imaging system 100. The usable length of catheter 102 (for example, the portion that can be inserted into a patient, specifically the vessel of interest) can be any suitable length and can be varied depending upon the application. Proximal end portion 114 of sheath 110 and proximal end portion 118 of imaging core 112 are connected to PIM 104. Proximal end portions 114, 118 are fitted with a catheter hub 124 that is removably connected to PIM 104. Catheter hub 124 facilitates and supports a rotational interface 182 that provides electrical and mechanical coupling between catheter 102 and PIM 104.

Distal end portion 120 of imaging core 112 includes a transducer assembly 122. In some embodiments, transducer assembly 122 is configured to be rotated (either by use of a motor or other rotary device, or manually by hand) to obtain images of the vessel. Transducer assembly 122 can be of any suitable type for visualizing a vessel and, in particular, a stenosis in a vessel. In the depicted embodiment, transducer assembly 122 includes a piezoelectric micro-machined ultrasonic transducer (“PMUT”) and associated circuitry, such as an application-specific integrated circuit (ASIC). An exemplary PMUT used in IVUS catheters may include a polymer piezoelectric membrane, such as that disclosed in U.S. Pat. No. 6,641,540, and co-pending applications entitled “Method of Fabricating a MEM's FACT Transducer,” Ser. No. 61/740,998 filed as attorney docket No. 44755.1062, “Focused Rotational IVUS Transducer Using Single Crystal Composite Material,” Ser. No. 61/745,425 filed as attorney docket No. 44755.931, and “Transducer Mounting Arrangements and Associated Methods for Rotational Intravascular Ultrasound (IVUS) Devices,” filed as attorney docket No. 44755.960 on even date herewith, each hereby incorporated by reference in its entirety. The PMUT may provide greater than 100% bandwidth for optimum resolution in a radial direction, and a spherically-focused aperture for optimum azimuthal and elevation resolution. Thus, transducer assembly 122 may provide a focused ultrasonic beam having a spot size of about 50 μm or less.

In some embodiments transducer assembly 122 may include a plurality of stationary components disposed around the circumference of distal end 120 of catheter 102. In such configuration, the components in transducer 122 may be piezo-electric elements distributed to form a phased-array configuration. The piezo-electric elements may be ceramic-based or polymer-based. Furthermore, in some embodiments the plurality of stationary components in transducer 122 may be configured to produce a focused acoustic impulse. In such embodiments, the stationary components produce an acoustic impulse according to a pre-selected excitation phase for each of the components.

Transducer assembly 122 may also include a housing having the PMUT and associated circuitry disposed therein. In some embodiments the housing has an opening that ultrasound signals generated by the PMUT transducer travel through. Alternatively, transducer assembly 122 includes a capacitive micro-machined ultrasonic transducer (“CMUT”). In yet another alternative embodiment, the transducer assembly 122 includes an ultrasound transducer array (for example, arrays having 16, 32, 64, or 128 components are utilized in some embodiments).

In some embodiments, a rotation of imaging core 112 within sheath 110 is controlled by PIM 104. For example, PIM 104 provides user interface controls that can be manipulated by a user. In some embodiments PIM 104 may receive, analyze, and/or display information received through imaging core 112. It will be appreciated that any suitable functionality, controls, information processing and analysis, and display can be incorporated into PIM 104. Thus, PIM 104 may include a processor circuit 154 and a memory circuit 155 to execute operations on catheter 102 and receive, process, and store data from catheter 102. In some embodiments PIM 104 receives data associated to ultrasound signals (echoes) detected by imaging core 112. PIM 104 processes the data and forwards the processed echo data to control system 106. Control system 106 may include a processor circuit 156 and a memory circuit 157 to execute operations on catheter 102 and receive, process, and store data from catheter 102. In some embodiments, PIM 104 performs preliminary processing of the echo data prior to transmitting the echo data to control system 106. PIM 104 may perform amplification, filtering, and/or aggregating of the echo data, using processor circuit 154 and memory circuit 155. PIM 104 can also supply high- and low-voltage DC power to support operation of catheter 102 including circuitry within transducer assembly 122.

In some embodiments, wires associated with IVUS imaging system 100 extend from control system 106 to PIM 104. Thus, signals from control system 106 can be communicated to PIM 104 and/or vice versa. In some embodiments, control system 106 communicates wirelessly with PIM 104. Similarly, it is understood that, in some embodiments, wires associated with IVUS imaging system 100 extend from control system 106 to monitor 108 such that signals from control system 106 can be communicated to monitor 108 and/or vice versa. In some embodiments, control system 106 communicates wirelessly with monitor 108.

Piezoelectric micro-machined ultrasound transducers (PMUTs) fabricated using a polymer piezoelectric material for use in transducer assembly 122, such as disclosed in U.S. Pat. No. 6,641,540 that is hereby incorporated by reference in its entirety, offer greater than 100% bandwidth for optimum resolution in the radial direction, and a spherically-focused aperture for optimum azimuthal and elevation resolution.

FIG. 1A illustrates a 3-dimensional (3D) Cartesian coordinate system XYZ oriented such that the Z-axis is aligned with the LA. In further descriptions of embodiments disclosed herein, a reference to a Cartesian plane or coordinate may be made in relation to FIG. 1. One of ordinary skill will recognize that the particular choice of coordinate axes in FIG. 1A is not limiting of embodiments as disclosed herein. The choice of coordinate axes is done for illustration purposes only.

FIG. 1B is a cross-sectional side view of a distal portion of a catheter used in an IVUS imaging system, according to some embodiments. In particular, FIG. 1B shows an expanded view of aspects of the distal portion of imaging core 112. In this exemplary embodiment, imaging core 112 is terminated at its distal tip by a housing 126 having a rounded nose and a cutout 128 for the ultrasound beam 150 to emerge from the housing. In some embodiments, a flexible driveshaft 132 of imaging core 112 is composed of two or more layers of counter wound stainless steel wires, welded, or otherwise secured to housing 126 such that rotation of the flexible driveshaft also imparts rotation to housing 126. In the illustrated embodiment, a PMUT MEMS transducer layer 121 includes a spherically focused portion facing cutout 128. In some embodiments, transducer assembly 122 may include application-specific integrated circuit (ASIC) 144 within distal portion 120 of imaging core 112. ASIC 144 is electrically coupled to transducer layer 221 through two or more connections.

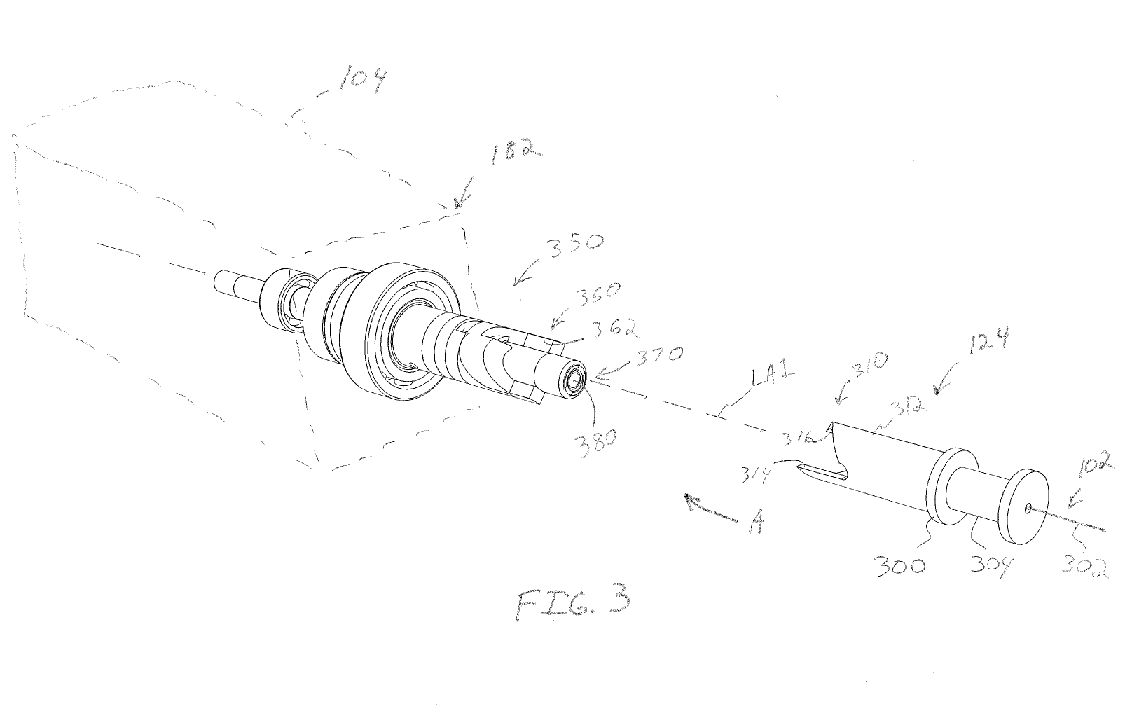

In some embodiments of the present disclosure ASIC 144 may include an amplifier, a transmitter, and a protection circuit associated with PMUT MEMS layer 121. In some embodiments, ASIC 144 is flip-chip mounted to a substrate of the PMUT MEMS layer 121 using anisotropic conductive adhesive or suitable alternative chip-to-chip bonding method. When assembled together PMUT MEMS layer 121 and ASIC 144 form an ASIC/MEMS hybrid transducer assembly 122 mounted within housing 126. An electrical cable 134 having four electrical conductors with optional shield 136 may be attached to transducer assembly 122 with solder 140. Electrical cable 134 may extend through an inner lumen of the flexible driveshaft 132 to proximal end 118 of imaging core 112. In proximal end 118, cable 134 is terminated to an electrical connector assembly 124 which may be joined to rotational interface coupling 182 of PIM 104 (cf. FIG. 3). In the illustrated embodiment, transducer assembly 122 is secured in place relative to the housing 126 by an epoxy 148 or other bonding agent. Epoxy 148 may serve as an acoustic backing material to absorb acoustic reverberations propagating within housing 126 and as a strain relief for the electrical cable 134 where it is soldered to transducer assembly 122.

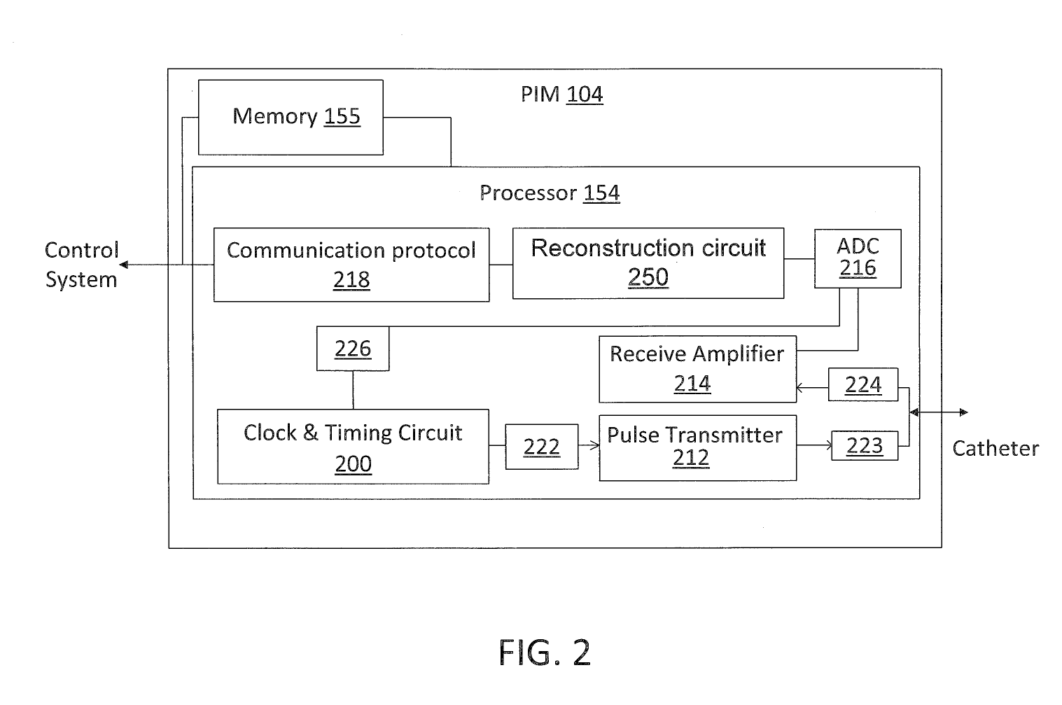

FIG. 2 is a block diagram of a Patient Interface Module (PIM) 104 for use in an IVUS imaging system, according to some embodiments. PIM 104 includes processor circuit 154 and memory circuit 155, described in detail above in relation to FIG. 1. PIM 104 provides a control signal 223 to a catheter, and receives data 224 from the catheter (e.g., catheter 102, FIG. 1). Control signal 223 may include a sequence of voltage pulses creating an acoustic impulse from a transducer assembly (e.g., transducer assembly 122). In some embodiments, control signal 223 is generated in a pulse transmitter 212 included in processor circuit 154. In some embodiments, each pulse from a plurality of pulses may include a single cycle of a signal having a selected frequency. In such embodiments, the frequency spectrum of such a pulse will be a signal centered at the selected frequency, having a bandwidth. Accordingly, pulse transmitter 212 may be configured to generate a plurality of voltage pulses centered at a plurality of frequencies. For example, the plurality of center frequencies for pulses provided by pulse transmitter 212 may include different frequencies, such as baseband frequencies and their harmonics. Thus, according to some embodiments, pulse transmitter circuit 212 has a transmission band which may include multiple center frequencies for a plurality of pulses provided to a transducer assembly.

In some embodiments, data 224 includes electrical signals received from catheter 102 and amplified by receive amplifier 214. The electrical signals in data 224 may be voltage signals. According to some embodiments, data 224 is an analog signal associated to an ultrasonic echo from a tissue structure around the transducer assembly. Analog-to-digital converter (ADC) 216 converts amplified electrical signal 224 into a digital signal. In some embodiments, the digital signal from ADC 216 is further processed by a reconstruction circuit 250. In some embodiments, data 224 includes voltage signals produced by the transducer assembly upon receiving an ultrasound echo signal from a tissue structure. The tissue structure may be surrounding a distal end of a catheter that includes the transducer assembly (e.g., distal end 120, cf. FIG. 1). The voltage signal in data 224 may include tissue responses at a plurality of frequencies, forming a reception band. Thus, receive amplifier 214 may include filters that produce a bandwidth including the reception band. Accordingly, in some embodiments the filtering of incoming data 224 and outgoing control signal 223 may be performed by ASIC 144 at distal end portion 120 of catheter 102 (cf. FIG. 1B).

Reconstruction circuit 250 may perform operations on the digitized, amplified data 224 such as data smoothing, averaging, noise filtering, and data interpolation. Thus, in some embodiments reconstruction circuit 250 may prepare the data provided by transducer assembly 122 for an image rendition of the tissue surrounding distal end 120 of catheter 102. The reconstructed digital data is transferred out of PIM 104 to IVUS control system 106 by a communication protocol circuit 218.

In some embodiments, a clock and timing circuit 200 provides a digitizing signal 226 to ADC 216, and transmitter timing signal 222 to pulse transmitter 212. According to some embodiments, clock and timing circuit 200 provides transmitter timing signal 222 and digitizing signal 226 using a common stable system clock. Some embodiments may include a phase-locked loop circuit in clock and timing circuit 200 to synchronize transmitter timing signal 222 and digitizing signal 226. In some embodiments transmitter timing signal 222 and digitizing signal 226 have the same phase, or their relative phase is fixed in time to within the resolution of clock and timing circuit 200.

In the present disclosure, is provided a new male PIM connector adapted for an IVUS catheter having four signal leads, which is developed for advancing the performance of rotational IVUS imaging catheters. Further provided is a dual compatible female PIM connector that can be connected either to a traditional two-electrical-contacts male connector that extends from a conventional two signal lead IVUS catheter, or to a four-electrical-contacts male connector that extends from a four signal lead IVUS catheter.

The conventional IVUS catheter utilizing two signal leads (or coaxial electrical cable) uses a standard ‘Suria’ connector that has only two electrical contacts on each part of the male and female connectors. The conventional two-electrical-contacts connectors cannot support the technology of the four signal leads IVUS catheters and provide a desired connection to a PIM. Therefore, if a dual-compatible PIM connector, especially a female connector, that can work on both types of IVUS catheters, the two signal leads IVUS catheters and the four signal leads IVUS catheters, and the same PIM, can be provided. Although not illustrated in the drawings, a catheter male connector 124 may include an inwardly facing projection on the assembly 312 that prevents it from mating with existing ‘Suria’ type female connectors as the lack of four connections would not allow the catheter to operate properly.

Referring now to FIG. 3, there is shown the catheter connection assembly 124 separated from the catheter coupling assembly 182 of the PIM 104 (shown in dashed lines). The catheter connection assembly 124 includes a body 300 having the proximal catheter portion 302 extending into a distal portion thereof. A gripping area 304 is formed between two shoulders. A rotational drive mechanism 310 includes an enlarged tubular member 312 having at least two drive dogs 314 and 316 formed on the proximal end thereof.

The PIM 104 likely includes a moveable sled portion (not shown) that allows the rotating transducer to be pulled back with respect to an outer sheath of the catheter. The PIM coupling 182 includes a catheter engagement assembly 350 having a drive assembly 360 and an electrical connection assembly 370. The drive assembly includes an enlarged cylindrical body having a pair of drive dogs 362 configured to mate with complimentary drive dogs 314 formed on the catheter proximal end. The electrical connection assembly 370 includes an internal passage 380 adapted to receive a corresponding protrusion extending from the proximal portion of the catheter. It will be appreciated that the catheter connection assembly 124 can be aligned by the user with the longitudinal axis LA1 of the PIM connection assembly 182 and then advanced in the direction of arrow A until the components are physically in intimate contact. As will be described further below, this coupling action not only establishes a rotational drive coupling, but also creates four separate electrical connections between the components.

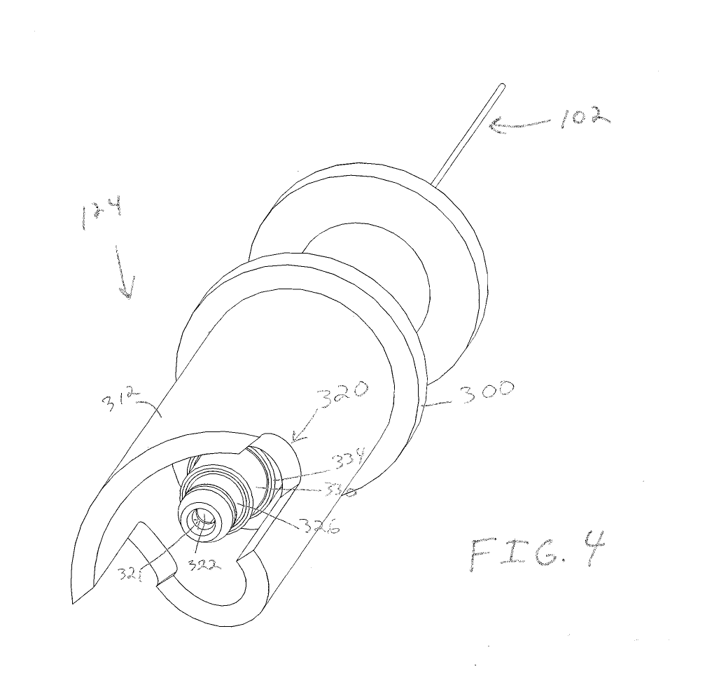

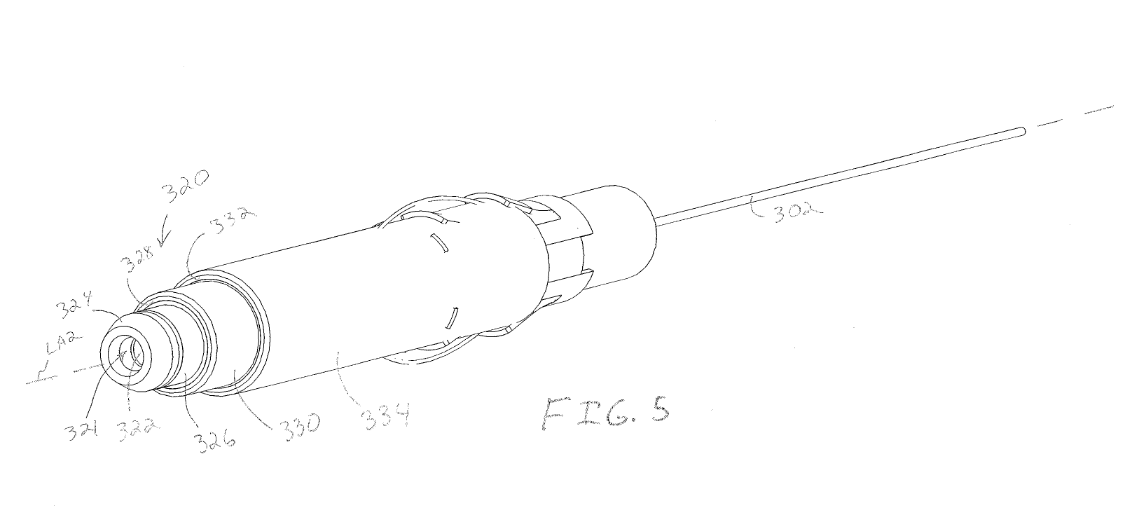

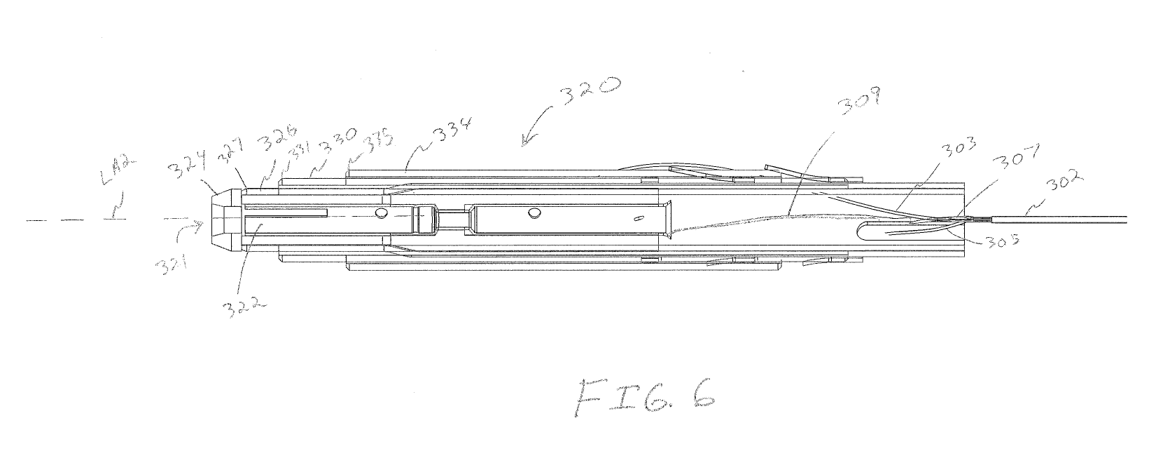

Referring now to FIGS. 4-6, there is shown additional detail of the catheter connection assembly 124. FIG. 4 shows a perspective view of the assembly with the projecting male connector 320 illustrated. FIG. 5 illustrates the male connector 320 with the outer rotational drive assembly 312 removed for ease of illustration. FIG. 6 is a cross-sectional side view of the male connection assembly shown in FIG. 5. The male connector 320 includes a central passage 321 aligned along longitudinal axis LA2. The central passage is defined by the inner surface of inner tubular conductive member 322. As shown in FIG. 6, the inner conductive tube or sleeve, 322 can include one or more slots to provide some strain relief to encourage a tight, flexible connection with a protruding pin of the female connector discussed below. Proximal tip 324 is formed of a non-conductive material on the extreme proximal end of the connection assembly. A second conductive tubular shell 326 having a first larger outside diameter is coaxially positioned about the first shell 322 and electrically spaced therefrom. The proximal end 327 of shell 326 extends adjacent proximal tip 324. A third conductive tubular shell 330 having a second larger outside diameter is coaxially positioned about the second shell 326 with its proximal end 331 longitudinally offset distally from the proximal end 327 of the second shell 326. Finally, outer most conductive shell 334 having a third larger outside diameter is coaxially positioned about third shell 330 with its proximal end 335 longitudinally offset distally from the proximal end 331 of the third shell 330. Each of the conductive sleeves is joined to one of the four electrically conductive wires 303, 305, 307, and 309 exiting imaging catheter 302. As best shown in FIG. 5, the exposed portions of shells 326, 330 and 334 create circumferential bands of conductive material that may be engaged to establish an electrical connection. Since each band has progressively larger outside diameters and have proximal ends longitudinal offset to form a telescoping configuration, the conductive bands define a stair stepped connection assembly that can also be referred to as a wedding cake stacked connection configuration. While the three outer bands are preferably engaged by contact with their outer surface, the fourth inner conductor is engaged by contact with the inner surface of member 322. Each conductive layer of the stepped connection assembly is electrically isolated from the adjacent member by an insulating layer or material. While the illustrated embodiment shows three outer shells it is contemplated that only two outer shells may be provided or that more than three shells may be provide with successive shells being coaxially positioned and longitudinally offset to form additional bands or steps of a stepped connection assembly.

FIG. 5 is a side perspective view of a four-electrical-contacts male connector 320, made according to an embodiment of the present disclosure, which can used for connecting a rotational IVUS catheter having four electrical signal leads to a PIM connector. The main body of the male connector 320 comprises three elongated, hollow tubular shells or bands. In an embodiment demonstrated in the referenced figure, FIG. 5, the elongated, hollow tubular shells have a configuration of a cylindrical shell with a circular cross-section. But different hollow tubular shells having a different cross section configuration such a square, a rectangle, an oval, or other polygons are also within the contemplation of the present disclosure.

To form the electrical contacts, in one embodiment, the whole outer surfaces of the three shells 326, 330, 334, and the whole inner surface of the innermost shell 322 that defined the cavity 321 may be made conductive. Or in another embodiment, only portions of those surfaces that would be directly engaged with the conductive surfaces of a female connector may be made conductive. For a practical purpose, the entire shells 326, 330, 334 may be made of a conducting material in one embodiment, or in another, the shells may have separate conductive layers formed on those referred surfaces where the four electrical contacts are to be formed. The conducting material may be a common material such as BeCu. But it could be any other conducting material known and used in the art.

The four electrical contacts to be formed on the three stepped bands, 326, 330, 334, and the inner surface portion of the innermost shell 322 should be insulated from one another. For that purpose, in one embodiment, the male connector 320 may further have thin cylindrical dielectric layers 328 and 332 concentrically disposed between the shells 326 and 330 as shown in FIG. 5 of the male connector 320 made according to an embodiment of the present disclosure. In an embodiment where only portions of the outer or inner surfaces of the shells are made conductive, the cylindrical dielectric shells may be disposed accordingly only between the conductive portions. For electrical insulation between the two electrical contacts formed on the inner and outer surfaces of the innermost shell 322 may be also effectuated by use of a suitable dielectric layer between the two surfaces. The dielectric layers may be made of any suitable dielectric materials known in the art.

Although the catheter having the male connector illustrated in FIGS. 4-6 is designed to support four electrical contacts coupling, the inventive concept of the present disclosure is not so limited. For instance, the same concept of a stepped banded design may apply to designing a male connector having six or even more electrical contacts, if it is demanded by further development of more advanced circuit architecture. More electrical contacts coupling can be provided by building more layers or shells of stepped bands around the three stepped banded structures shown in FIGS. 4-6 by slightly increasing the diameter of the rotational housing 312 and the catheter housing, if used. Therefore, if the sizes of the housing are not constraints, then theoretically, there is no limit to the number of contacts to be formed in the current approach of a stepped banded design in the present disclosure.

As briefly described before, the catheter 302 connected to the male connector 320 has an electrical cable of four conducting lines corresponding to four signal leads for transmitting and receiving signals and DC power from the PIM. The four wire electrical interface offers a wide array of benefits, with minimal compromise to the performance of the circuit and transducer, while maintaining a small cable dimension that can be readily accommodated by a rotational IVUS catheter. The four conducting lines may be twisted together into a symmetrical quad and treated as two diagonal conductor pairs. In one implementation, the four signals leads may be designated as PIM+, PIM−, HV (high voltage), and GND (ground), where the PIM+/− conductor pair lines may carry various digital signals from the PIM to an application-specific integrated circuit (ASIC) in the form of a balanced differential signal pair, while the HV signal line carries a high voltage DC supply to the ASIC, and to power the transmit circuitry. The catheter using four signal leads circuit architecture may provide images of high resolution in all three dimensions owing to its focused aperture and wide bandwidth, enable driving of a signal over a long transmission line, and overcome the low transmit efficiency of the polymer piezoelectric. Further, the circuit architectures using four signal leads may ensure efficient delivery of the received ultrasound echo signal from the transducer back to the PIM and IVUS processing components, while enabling delivery of high voltage pulses directly to the transducer without significant cable losses experienced in a conventional two signal leads rotational IVUS system.

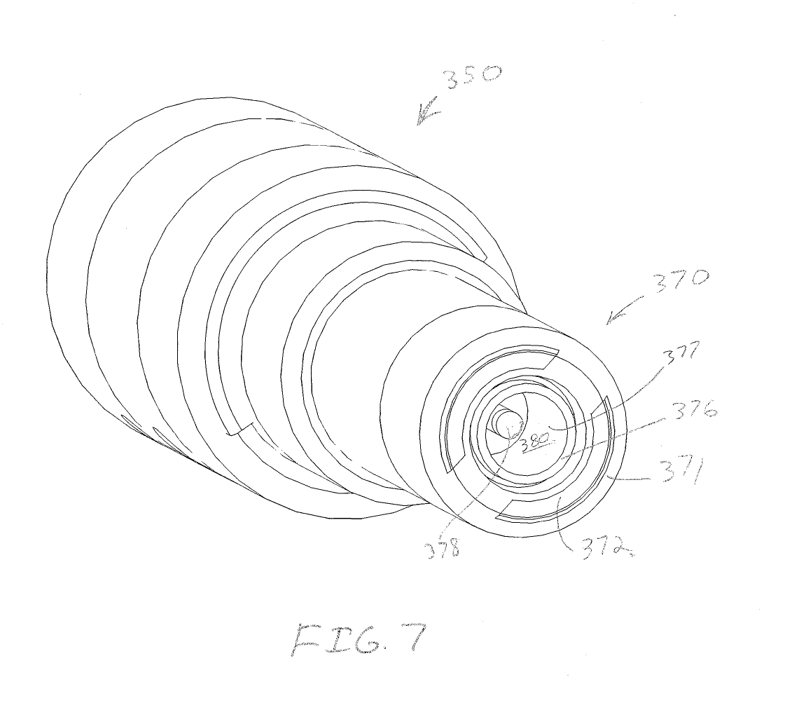

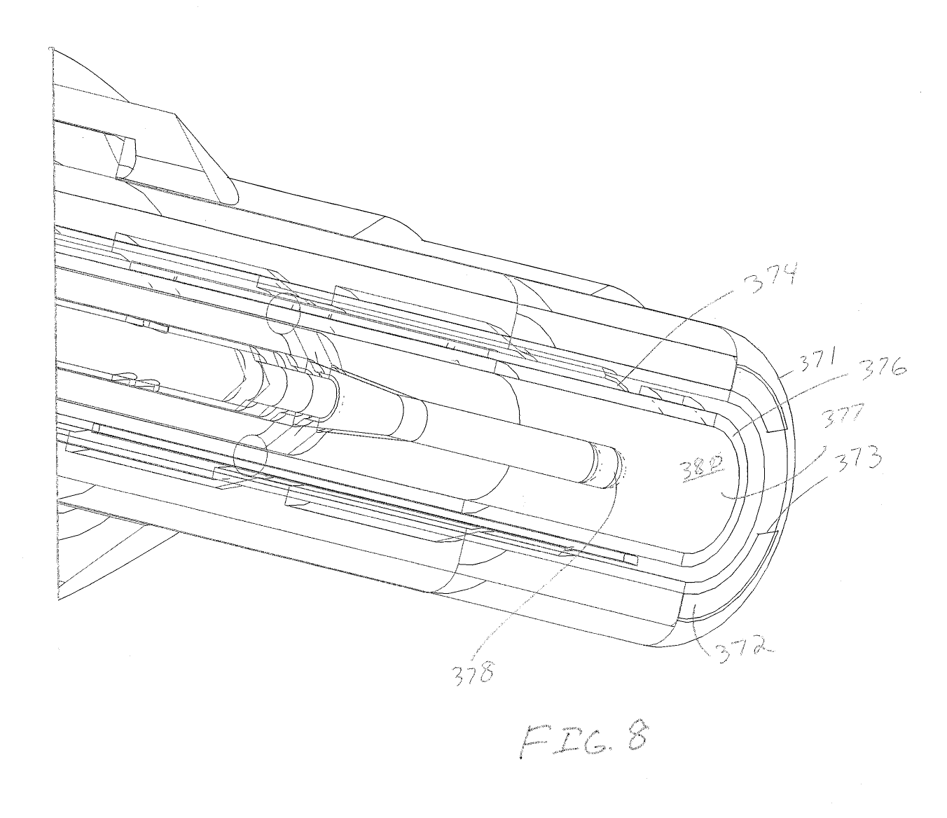

Referring now to FIGS. 7 and 8, there is shown a perspective side view and perspective cross-section view of a four-electrical-contacts female connector 350, made according to an embodiment of the present disclosure. The four-electrical-contacts female connector 370 is designed to be dual-compatible, that is, capable of mating, and being electrically coupled with both a conventional two-electrical-contacts male connector and a four-electrical-contacts male connector, as shown in FIGS. 4-6.

The dual-compatible four-electrical-contacts female connector 350 has a proximal portion supporting the drive assembly 360 and a distal portion adjacent the distal end 371, the distal portion including the female electrical connector assembly 370. The connection assembly 370 has a central shaft electrode 378 and three elongated, hollow tubular shells, 372, 374 and 376. In an embodiment demonstrated in the referenced figures, FIGS. 7 and 8, the elongated, hollow tubular shells have a configuration of a cylindrical shell having a circular cross-section in order to mate with a male connector having cylindrical mating parts such as shown in FIG. 4. But since the male connector 300 could have different hollow tubular shells of a different cross sectional configuration other than a circle in the present disclosure, the female connector 370 as well is designed to have a different size and configuration in accordance with the change of the mating male connector in size and configuration.

The three cylindrical shells, 372, 374 and 376, are spaced-apart and concentrically and sequentially disposed around one another along the longitudinal axis of the female connector 350. At a center of the coaxial cylindrical shells, 372, 374 and 376, cylindrical shaft 378 extends linearly along the longitudinal axis from the proximal portion of the connector and is positioned within the inner surface 377 of shell 376. This cylindrical shaft 378 is sized to be inserted into the cylindrical cavity 321 of the four-electrical-contacts male connector 310 for electrically coupling with the interior surface of the electrically conductive sleeve 322. The shaft 378 is sized and configured to be frictionally engaged with the inner surface of the cylindrical sleeve 322 sufficiently intimately to establish electrical coupling. One of the four electrical contacts formed on the female connector 350 is formed on the outer surface of the shaft 378. In one embodiment, the entire outer surface of the shaft 378 may be made conductive, but in another embodiment, only a portion of the outer surface of the portion of the shaft 378 may be made conductive. In the latter case, the position and size of the conductive portion needs to be configured such that when mated with male connector 310, the conductive portion may mate with the mating electrical contact formed on the inner surface of the sleeve 322.

The three cylindrical shells, 372, 374, and 376, are spaced-apart by insulating materials and concentrically and sequentially disposed around one another and around the central shaft 378. Out of the four electrical contacts to be formed on the female connector 350, the three remaining electrical contacts are formed on respective inner surfaces of the three cylindrical shells, 372, 374, and 376 the respective proximal ends. To form the electrical contacts, in one embodiment, the whole inner surfaces of the three shells 372, 374, and 376, may be made conductive. Or in another embodiment, only portions of those surfaces that would be directly engaged with the conductive surfaces of the male connector 300 may be made conductive. For a practical purpose, the entire shells 372, 374, and 376, themselves, may be made of a conducting material in one embodiment, or in another, the shells may have separate conductive layers formed on those referred surfaces at points where the four electrical contacts are to be formed. The conducting material may be a common material such as BeCu. But it could be any other conducting material known and used in the art.

The three cylindrical shells, 372, 374, and 376, are sized, positioned, and configured such that their three respective inner surfaces can mate, intimately and tightly, with the three respective stepped bands, 326, 330, and 334 of the four-electrical-contacts male connector 320. More particularly, each cylindrical shell is sized to provide an intimate contact between its inner surface with the outer surface of the mating stepped band. Further, the three cylindrical shells, 372, 374, and 376, are sized, positioned, and configured such that the three female electrical contacts formed on the respective inner surfaces can be electrically coupled with the three respective male electrical contacts respectively formed on the stepped bands, 326, 330, and 334 of the four-electrical-contacts male connector 320. The configurations to be determined for the three cylindrical shells, 372, 374, and 376, in consideration of the size and configurations of a mating male connector, may include not only their shape, which, in the illustrated embodiments is a cylindrical shell. They may include also the mutual radial distances among the individual shells, the thicknesses of individual shells, the lengths of individual shells, and the positions of individual shells relative to one another and relative to the shaft 378. For instance, in one embodiment, the innermost cylindrical shell 376 is the most recessed from the proximal end 371 out of the three shells in order to mate with the first stepped band 326 of the male connector 320, which is the most protruding outward among the three stepped bands. Similarly, the middle shell 374 and the outermost shell 372 are sequentially less recessed than the innermost shell 376 in order to sequentially and accurately mate with the second and third stepped bands, 330 and 3334, of the male connector 320, respectively. In the alternative form illustrated in FIGS. 7 and 8, the innermost shell 376 is moveable from the extended position shown to a retracted position where the distal end of the shell 376 is positioned proximally of the distal end of shell 374. As the male member 320 is inserted into the cavity 380, the shoulder formed by the insulating material 328 and band 330 will push the shell 376 proximally within the female housing. With this particular manner of a stepped contact design, the electrical contacts formed in the male connector 300 would not have to run over each of the electrical contacts on the female connector 500 before reaching their right respective mating contacts. The stepped contact design in the present disclosure, therefore, provides the advantages of eliminating the possibility of shortening the wrong contact or pin, and extending the wear life of a connector. In addition, the position of the shell 376 around the pin 378 allows the female connector to be backward compatible with existing Suria type connectors having only two electrical connectors.

In one embodiment, in order to control the force of connection or engagement between the mating parts, the cylindrical shell and stepped band, for providing more intimate coupling, one or more of the three cylindrical shells, 376, 374, and 372 may be split at a portion to create slits or gaps 373 as shown in FIG. 8.

Still referring to FIG. 3, the distal end of the female connector 350 is connected and electrically coupled to a patient interface module (PIM). Rotation of the imaging core (and thus rotation of the transducer assembly) within the catheter 102 is controlled by the PIM as it receives user input either directly or from a console connected to the PIM which provides user interface controls that can be manipulated by a user.

Typically, a Radio-Frequency Identification (RFID) chip is used for a rotational a ultrasound transducer catheter having carrying two signal leads to assist in identifying the catheter type attached to the PIM as well as providing serial number information and/or calibration data. Therefore, the PIM connected to the dual-compatible female connector in the present disclosure would preferably include at least one RFID reader/writer. Further, a rotational catheter having carrying four signal leads may require additional components such as an Electrically Erasable Programmable Read-Only Memory (EEPROM) and or small magnets that may convey further information about the attached catheter. In one embodiment, the EEPROM may be located inside the plastic rotational assembly 312 with an additional electrical contact to read the EEPROM located on the drive assembly 360 for detecting insertion of the male connector into the female connector and reading the EEPROM information. Or in another embodiment, the EEPROM may reside on a printed circuit board (PCB) adjacent the RFID, and a connector from the PIM may be mated with the EEPROM PCB.

Also provided in the present disclosure is a method of providing electrical connection between a patient interface module (PIM) and an intravascular ultrasound (IVUS) device. As one step, it is provided either a two-electrical-contacts male connector having two mutually insulated male electrical contacts when the intravascular ultrasound (IVUS) device uses two-lead conducting lines for connection to the PIM, or a four-electrical-contacts male connector having four mutually insulated male electrical contacts when the intravascular ultrasound (IVUS) device uses four-lead conducting lines for connection to the PIM. Herein, the word ‘provide’ is used in a broad sense to encompass all modes of procuring an object, including, not limited to, ‘purchasing’, ‘preparing’, ‘manufacturing’, ‘arranging,’ or ‘making in order’ either the two-electrical-contacts male connector or the four-electrical-contacts male connector.

In an embodiment, the two-electrical-contacts male connector may include an elongated hollow tubular male body, and the two male electrical contacts may be respectively formed on inner and outer surfaces of the tubular male body.

Also, in an embodiment, the four-electrical-contacts male connector may include a three elongated hollow tubular male bodies, which are concentrically and sequentially disposed around one another in an expanded telescopic fashion so that the three respective outer surfaces may form a stepped banded structure. In the referenced embodiment, one of the four male electrical contacts may be formed on an inner surface of the innermost tubular male body, and the remaining three may be formed on the respective stepped banded outer surfaces of the three tubular male bodies. Also, the four-electrical-contacts male connector may further comprise four electric wires, which are electrically coupled, at first ends thereof, with the respective four male electrical contacts, and configured to be electrically coupled, at opposing second ends thereof, with a four-lead electrical cable of the intravascular ultrasound (IVUS) device. In one embodiment, the electrical insulation among the four electrical male contacts of the four-electrical-contacts male connector may be achieved by dielectric layers concentrically disposed among the three tubular male bodies. Also, in one embodiment, the three tubular male bodies may define similar stepped banded respective outer surfaces at their respective distal ends as well, respectively extending toward respective proximal ends.

At the next step, a four-electrical-contacts female connector having proximal and distal portions is provided. The proximal portion has four mutually insulated female electrical contacts, and is sized and configured to be connected and electrically coupled, either with a two-electrical-contacts male connector having two mutually insulated male electrical contacts via two of the four female electrical contacts, or with a four-electrical-contacts male connector having four mutually insulated male electrical contacts via the four female electrical contacts. More specifically, in an embodiment, the proximal portion of the female connector may comprise: three spaced-apart hollow tubular female bodies that are concentrically and sequentially disposed around one another; and a shaft disposed within the innermost tubular female body. The three female bodies are sized and configured such that respective inner surfaces thereof can mate with the respective three stepped banded outer surfaces of the four-electrical-contacts male connector. The shaft is sized and configured to mate with a central cavity defined by the innermost tubular male body. Further, for connection to the two-electrical-contacts male connector, the shaft and the innermost tubular female body are further sized and configured such that the inner surface of the innermost tubular female body and an outer surface of the shaft can mate, respectively, with the outer and inner surfaces of the elongated hollow tubular male body of the two-electrical-contacts male connector.

In an embodiment, one of the four female electrical contacts is formed on an outer surface of the shaft, and the rest are formed on the respective inner surfaces of the three tubular female bodies.

At the next step, the proximal portion of the female connector is connected, either with a proximal portion of the two-electrical-contacts male connector when the intravascular ultrasound (IVUS) device uses two-lead conducting lines for connection to the PIM, or with a proximal portion of the four-electrical-contacts male connector when the intravascular ultrasound (IVUS) device uses four-lead conducting lines for connection to the PIM. Finally, the catheter may be operated within a patient to obtain data that is transferred through the male/female connection.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An imaging catheter configured for insertion into the body, the catheter comprising:
a catheter body having a proximal portion and a distal portion, with a longitudinal axis extending therebetween;
a sensor assembly mounted on the distal portion;
at least three electrical conductors extending from the sensor assembly to the proximal portion;
a connection assembly positioned on the proximal portion receiving the electrical conductors, the connection assembly including:
at least two electrically conductive, elongated tubular male bodies having respective proximal and distal ends, the two tubular male bodies having different outer diameters and being coaxially positioned about the longitudinal axis with the proximal ends offset longitudinally, each of the two tubular male bodies having an exposed outer surface, a third electrically conductive member disposed adjacent the two tubular male bodies, the third electrically conductive member having an exposed inner surface, and insulating material electrically isolating each of the two tubular male bodies and the third conductive member which are each connected to one of the three electrical conductors.

2. The catheter of claim 1, further comprising a fourth electrical conductor extending from the sensing assembly and an additional electrically conductive male body coaxial disposed and longitudinal offset from the two elongate tubular bodies and being connected to the fourth conductor.

3. The catheter of claim 1, wherein the sensor is a piezoelectric ultrasound transducer.

4. The catheter of claim 2, wherein the sensor is a piezoelectric ultrasound transducer.

5. The catheter of claim 2, wherein the sensor is a polymer based focused piezoelectric ultrasound transducer.

6. The catheter of claim 2, wherein each of the three hollow tubular male bodies has a general outer configuration of a hollow cylindrical shell defining a stepped banded connector assembly.

7. The catheter of claim 6, further including a rotational drive member joined to the proximal portion of the catheter adjacent the stepped banded connector assembly.

8. The catheter of claim 7, wherein the stepped banded connector assembly is positioned inside of the rotational drive assembly.

9. A patient interface module having a female electrical connector for a patient interface module (PIM), comprising:
a proximal portion having four mutually insulated electrical contacts, the proximal portion being sized and configured to mate, and be electrically coupled, with a two-electrical-contacts male connector via two of the four electrical contacts, or with a four-electrical-contacts male connector via the four electrical contacts; and
a distal portion configured to be connected and electrically coupled to the patient interface module.

10. The female electrical connector of claim 9, wherein the proximal portion comprises:
three spaced-apart hollow tubular female bodies concentrically and sequentially disposed around one another, the three tubular female bodies being sized and configured such that three respective inner surfaces thereof can mate with three respective stepped banded outer surfaces of a four-electrical-contacts male connector, the outer surfaces being defined by disposing three elongated tubular male bodies concentrically and sequentially around one another in an expanded telescopic fashion; and
a shaft concentrically disposed within the innermost tubular female body,
the shaft being sized and configured to mate with an elongated cavity defined by an inner surface of the innermost tubular male body of the four-electrical-contacts male connector.

11. The female electrical connector of claim 10, wherein one of the four electrical contacts is formed on an outer surface of the shaft, and the rest are formed on the respective inner surfaces of the three tubular female bodies.

12. The female electrical connector of claim 10, wherein the shaft and the innermost tubular female body are further sized and configured such that the inner surface of the innermost tubular female body and the outer surface of the shaft can mate, respectively, with outer and inner surfaces of an elongated hollow tubular male body of a two-electrical-contacts male connector.

13. The female electrical connector of claim 10, wherein each of the three tubular female bodies has a general configuration of a hollow cylindrical shell.

14. The female electrical connector of claim 10, wherein one or more of the three tubular female bodies are slit at a portion thereof for providing intimate engagement with the male connector.

15. A method of providing electrical connection between a patient interface module (PIM) and an intravascular ultrasound (IVUS) device, comprising:
providing a two-electrical-contacts male connector having two mutually insulated male electrical contacts when the intravascular ultrasound (IVUS) device uses two-lead conducting lines for connection to the PIM, or a four-electrical-contacts male connector having four mutually insulated male electrical contacts when the intravascular ultrasound (IVUS) device uses four-lead conducting lines for connection to the PIM;
providing a female connector having proximal and distal portions, the proximal portion having four mutually insulated female electrical contacts and being sized and configured to be connected and electrically coupled with a two-electrical-contacts male connector having two mutually insulated male electrical contacts via two of the four female electrical contacts, or with a four-electrical-contacts male connector having four mutually insulated male electrical contacts via the four female electrical contacts;
connecting the proximal portion of the female connector with a proximal portion of the two-electrical-contacts male connector when the intravascular ultrasound (IVUS) device uses two-lead conducting lines for connection to the PIM, or with a proximal portion of the four-electrical-contacts male connector when the intravascular ultrasound (IVUS) device uses four-lead conducting lines for connection to the PIM; and
electrically coupling the distal portion of the female connector to the PIM.

16. The method of claim 15, wherein the two-electrical-contacts male connector includes an elongated hollow tubular male body, and the two male electrical contacts are respectively formed on inner and outer surfaces of the tubular male body, and the four-electrical-contacts male connector includes three elongated hollow tubular male bodies concentrically and sequentially disposed around one another in an expanded telescopic fashion to define three stepped banded respective outer surfaces, and one of the four male electrical contacts is formed on an inner surface of the innermost tubular male body, and the rest are formed on the respective stepped banded outer surfaces of the three tubular male bodies.

17. The method of claim 16, wherein the proximal portion of the female connector comprises three spaced-apart hollow tubular female bodies concentrically and sequentially disposed around one another and a shaft disposed within the innermost tubular female body, the three female bodies being sized and configured such that respective inner surfaces thereof can mate with the respective three stepped banded outer surfaces of the three tubular male bodies, and the shaft being sized and configured to mate with a central cavity defined by the innermost tubular male body.

18. The method of claim 17, wherein one of the four female electrical contacts is formed on an outer surface of the shaft, and the rest are formed on the respective inner surfaces of the three tubular female bodies.

19. The method of claim 17, wherein the shaft and the innermost tubular female body are further sized and configured such that the inner surface of the innermost tubular female body and an outer surface of the shaft can mate, respectively, with the outer and inner surfaces of the elongated hollow tubular male body of the two-electrical-contacts male connector.

20. The method of claim 15, further comprising connecting and electrically coupling a distal portion of the two-electrical-contacts male connector or the four-electrical-contacts male connector to the intravascular ultrasound (IVUS) device.

21. The method of claim 21, wherein the intravascular ultrasound (IVUS) device is a piezoelectric micromachined ultrasound transducer (PMUT) rotational catheter.

22. The method of claim 16, wherein the four-electrical-contacts male connector further comprises four electric wires electrically coupled, at first ends thereof, with the respective four male electrical contacts, and configured to be electrically coupled, at opposing second ends thereof, with a four-lead electrical cable of the intravascular ultrasound (IVUS) device.

23. The method of claim 16, wherein the electrical insulation among the four electrical contacts of the four-electrical-contacts male connector is achieved by dielectric layers concentrically disposed among the three tubular male bodies.

24. The method of claim 16, wherein the three tubular male bodies define stepped banded respective outer surfaces at the respective distal ends as well, respectively extending toward respective proximal ends.
